(19) United States
(12) Patent Application Publication
(10) Pub. No.: US 2014/0316233 A1
(43) Pub. Date: Oct. 23, 2014

(54) NON-CONTACT TONOMETER

(71) Applicant: CANON KABUSHIKI KAISHA, Tokyo (JP)

(72) Inventors: Shintaro Akiba, Isehara-shi (JP); Yasuhiro Dobashi, Matsudo-shi (JP)

(73) Assignee: CANON KABUSHIKI KAISHA, Tokyo (JP)

(21) Appl. No.: 14/252,951

(22) Filed: Apr. 15, 2014

(30) Foreign Application Priority Data
Apr. 17, 2013 (JP) 2013-086792

(51) Int. Cl. A61B 3/16

(52) U.S. Cl. CPC A61B 3/165
USPC 600/401

(57) ABSTRACT

In a non-contact tonometer, puffing of air unnecessary for measurement of an eye to be inspected after driving of a solenoid is stopped is suppressed. In the non-contact tonometer including a corneal shape change unit configured to change the shape of the cornea by pressurizing and supplying a gas in a cylinder by a piston, and an eye pressure measuring unit configured to measure the eye pressure from the state of the shape change of the cornea, an opening portion configured to be formed in the outer wall of the cylinder and decide the internal volume of the cylinder when pressurizing the gas, and a pressurized gas volume change unit configured to change a position where the opening portion can connect the inside of the cylinder with the outside, and change the internal volume of the cylinder when pressurizing the gas.

### BACKGROUND OF THE INVENTION

1. Field of the Invention

The present invention relates to a non-contact tonometer which computes an eye pressure value from a corneal shape change signal obtained by an optical detection means upon puffing air against an eye to be inspected to change the corneal shape.

2. Description of the Related Art

A non-contact tonometer is typified by an air puff tonometer developed by Bernard Grolman. This tonometer optically detects the applanation of the cornea by puffing air against the cornea of an eye to be inspected from a nozzle about 11 mm apart from the cornea. Then, the time till the applanation is calibrated by a contact Goldmann tonometer, computing an eye pressure value.

Most tonometers of this type use a system in which a piston moves inside a cylinder connected to an air puff nozzle portion to pressurize air in the cylinder and puff the air from the nozzle. As the piston drive system, a solenoid is generally used because of a high initial torque and a simple arrangement.

Non-contact tonometers are requested to have a wide measurement range from a low eye pressure to a high eye pressure arising from a disease such as glaucoma. To measure a high eye pressure, a sufficient amount of air needs to be puffed against an eye to be inspected, and the cylinder volume is designed with reference to the high eye pressure. For an eye to be inspected with a low eye pressure, the amount of air puffed is adjusted by changing the drive current or drive time of the solenoid in accordance with the eye pressure value of the eye.

The system using the solenoid is inexpensive and simple in arrangement, but is known to have several demerits. The solenoid has a simple structure constituted by only a winding and permanent magnet, operates in only one direction, and thus needs to use a return system such as a return spring.

In general, the actuating force of the solenoid is much larger than that of the return spring. Once the solenoid is energized to drive the piston, inertial force generated by the weight of the piston acts even after the current is stopped. This makes it difficult to stop the piston at a target position.

Particularly when measuring an eye to be inspected with a low eye pressure, the amount of air necessary for applanation is small, and the piston needs to be stopped at a considerably early stage with respect to the piston drive range in the cylinder. However, owing to the inertial force of the piston, unnecessary air is puffed against the eye, annoying the object.

To solve the above problem, Japanese Patent Application Laid-Open No. H09-201335 discloses 1) an invention in which the amount of movement by the inertial force after interruption of the piston drive current is decreased by increasing at a gradual rise rate a drive voltage applied to a solenoid which drives a piston.

Further, Japanese Patent Application Laid-Open No. 2009-82514 discloses 2) a system which releases air through an electromagnetic valve to prevent puffing of pressurized air in a cylinder against an eye to be inspected. This invention adopts the system which releases air from the cylinder through the electromagnetic valve. In addition, this invention predicts the timing to open the electromagnetic valve from the first measurement in consideration of the response delay of the electromagnetic valve, and opens the electromagnetic valve at an appropriate timing to reduce unnecessary air puffed against an eye to be inspected.

Even in a circuit configured to gradually increase the rise rate of an applied voltage as in the invention disclosed in Japanese Patent Application Laid-Open No. H09-201335 described above, air puffing caused by the inertial force of the piston cannot be prevented. If the applied voltage is designed to be variable, the control circuit becomes complicated.

Even if the piston can be stopped suddenly by some kind of control system, pressurized air in the cylinder leaks out from a puffing nozzle because the pressure of this air is higher than the atmospheric pressure. Hence, the aforementioned invention does not lead to a solution for the fundamental problem that uncomfortable air is puffed against an object.

The method of releasing pressurized air from the cylinder by opening the electromagnetic valve, which is disclosed in Japanese Patent Application Laid-Open No. 2009-82514 described above, is theoretically effective. However, to instantaneously release pressurized air from the cylinder, the opening of the electromagnetic valve needs to be much larger than the nozzle, so a large electromagnetic valve is required. The large electromagnetic valve costs, and it is difficult to mount the large electromagnetic valve in a limited space inside the apparatus. This raises the hurdle for employing this method. Further, the time till the timing to open the electromagnetic valve after detecting a corneal shape change is as short as several ms. A control circuit for controlling the electromagnetic valve in such a short time becomes complicated and expensive.

### SUMMARY OF THE INVENTION

The present invention solves the above-described problems, and provides a non-contact tonometer capable of suppressing puffing of unnecessary air against an eye to be inspected by a low-cost, simple arrangement.

According to the present invention, there is provided a non-contact tonometer including a corneal shape change unit configured to change a shape of a cornea of an eye to be inspected by pressurizing a gas in a cylinder by a piston and puffing the pressurized gas from a nozzle against the cornea, the corneal shape change unit including an opening portion configured to be formed in an outer wall of the cylinder and decide an internal volume of the cylinder when pressurizing the gas, and an eye pressure measuring unit configured to detect a state of a shape change of the cornea and measure an eye pressure of the eye to be inspected, comprising a pressurized gas volume change unit configured to change a position where the opening portion can connect inside of the cylinder with outside, and change the internal volume of the cylinder when pressurizing the gas.

The non-contact tonometer according to the present invention can puff an optimum amount of air in accordance with an eye pressure value by changing the position of the opening portion formed in the outer wall of the cylinder. Even when the piston is controlled by driving the solenoid, puffing of air unnecessary for measurement caused by the inertial force of the piston can be prevented.

The non-contact tonometer according to the present invention can be configured by only adding the opening portion and opening portion position selection system to a conventional apparatus. Thus, a low-cost, compact apparatus can be provided.

Further features of the present invention will become apparent from the following description of exemplary embodiments with reference to the attached drawings.

### BRIEF DESCRIPTION OF THE DRAWINGS

FIG. 1 is a view showing the outer appearance of a non-contact tonometer.

FIG. 2 is a view showing the arrangement of the optical system of a measurement portion.

FIG. 3 is a view showing the outer appearance of the cylinder portion according to the first embodiment.

FIGS. 4A, 4B, 4C, 4D and 4E are views for explaining operations of a cylinder portion in respective stages according to the first embodiment.

FIG. 5 is a system block diagram according to the first embodiment.

FIGS. 6A, 6B and 6C are views for explaining an operation in eye pressure measurement in a related art.

FIG. 7 is a graph showing the relationship between a corneal shape change signal and a pressure signal in the related art.

FIGS. 8A, 8B and 8C are views for explaining an operation in eye pressure measurement according to the first embodiment.

FIG. 9 is a graph showing the relationship between a corneal shape change signal and a pressure signal according to the first embodiment.

FIG. 10 is a flowchart for explaining the first embodiment.

FIG. 11 is a view showing the outer appearance of a cylinder portion according to the second embodiment.

FIGS. 12A, 12B, 12C and 12D are views for explaining operations of the cylinder portion in respective stages according to the second embodiment.

FIG. 13 is a view showing the outer appearance of a cylinder portion according to the third embodiment.

FIGS. 14A, 14B, 14C and 14D are views for explaining operations of the cylinder portion in respective stages according to the third embodiment.

### DESCRIPTION OF THE EMBODIMENTS

Preferred embodiments of the present invention will now be described in detail in accordance with the accompanying drawings.

### First Embodiment

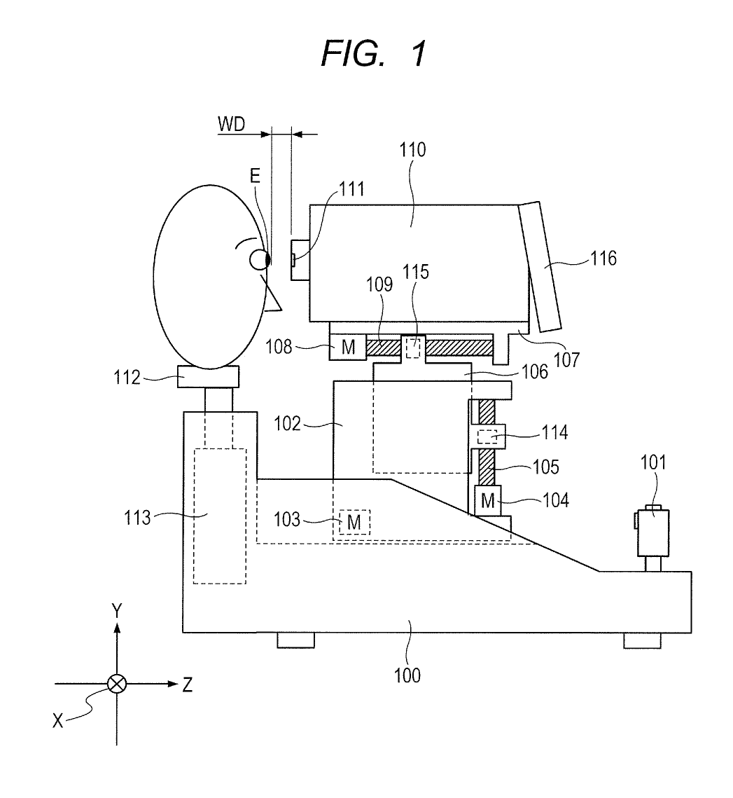

FIG. 1 is a view showing the schematic arrangement of a non-contact tonometer according to the present invention.

A frame 102 is movable in the left-and-right direction (to be referred to as an X axis direction hereinafter) with respect to a base 100. A drive system in the X axis direction is constituted by an X axis drive motor 103 fixed on the base 100, a feed screw (not shown) coupled to a motor output axis, and a nut (not shown) fixed to the frame 102 so as to be movable on the feed screw in the X axis direction. The motor 103 rotates to move the frame 102 in the X axis direction via the feed screw and nut.

A frame 106 is movable in the up-and-down direction (to be referred to as a Y axis direction hereinafter) with respect to the frame 102. A drive system in the Y axis direction is constituted by a Y axis drive motor 104 fixed on the frame 102, a feed screw 105 coupled to a motor output axis, and a nut 114 fixed to the frame 106 so as to be movable on the feed screw in the Y axis direction. The motor 104 rotates to move the frame 106 in the Y axis direction via the feed screw and nut.

A frame 107 is movable in the back-and-forth direction (to be referred to as a Z axis direction hereinafter) with respect to the frame 106. A drive system in the Z axis direction is constituted by a Z axis drive motor 108 fixed on the frame 107, a feed screw 109 coupled to a motor output axis, and a nut 115 fixed to the frame 106 so as to be movable on the feed screw in the Z axis direction. The motor 108 rotates to move the frame 107 in the Z axis direction via the feed screw and nut.

A measurement portion 110 is fixed on the frame 107 to perform measurement. The object-side end portion of the measurement portion is provided with a nozzle 111 for discharging air necessary for eye pressure measurement. The examiner-side end portion of the measurement portion 110 is provided with an LCD monitor 116 which is a display member for observing an eye E to be inspected.

The base 100 is provided with a joy stick 101 which is an operation member for positioning the measurement portion 110 with respect to the eye E to be inspected.

When measuring an eye pressure, the object rests his/her chin on a chin rest 112 and presses his/her forehead against the forehead rest portion of a face rest frame (not shown) fixed on the base 100, thereby fixing the position of an eye to be inspected. A drive system 113 can adjust the chin rest 112 in the Y axis direction in accordance with the face size of the object.

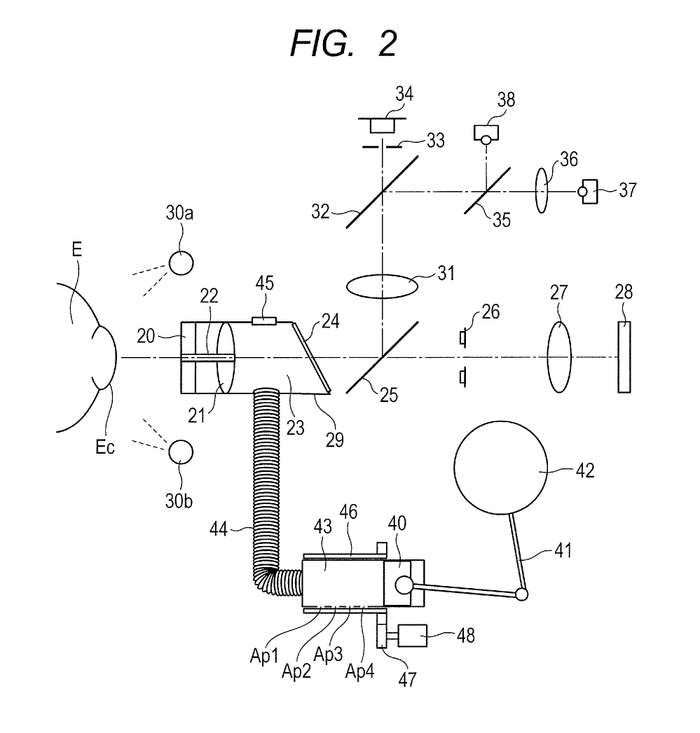

FIG. 2 is a view showing the arrangement of an optical system in the measurement portion 110. A nozzle 22 is arranged on the central axis of a plane parallel glass 20 and objective lens 21 so as to face a cornea Ec of the eye E to be inspected. An air chamber 23, observation window 24, dichroic mirror 25, prism stop 26, imaging lens 27, and CCD 28 are sequentially arrayed behind the nozzle 22. These building components constitute the light receiving optical path and alignment detection optical path of an observation optical system for the eye E to be inspected.

An objective lens barrel 29 supports the plane parallel glass 20 and objective lens 21. Fundus illumination light sources 30a and 30b for illuminating the eye E to be inspected are arranged outside the objective lens barrel 29.

For descriptive convenience, the fundus illumination light sources 30a and 30b are arranged in the vertical direction in FIG. 2. In practice, however, the fundus illumination light sources 30a and 30b are arranged to face each other with respect to the optical axis in a direction perpendicular to the plane of the drawing.

A relay lens 31, half mirror 32, aperture 33, and light receiving element 34 are arranged in the reflecting direction of the dichroic mirror 25. Note that the aperture 33 is arranged at a position where it becomes conjugate to a cornea reflection image of a measurement light source 37 (to be described later) when the cornea changes into a predetermined shape. The aperture 33 and light receiving element 34 constitute a shape change detection light receiving optical system used when the shape of the cornea Ec changes in the visual axis direction.

The relay lens 31 is designed to form a cornea reflection image almost equal in size to the aperture 33 when the cornea Ec changes into a predetermined shape.

A half mirror 35, a projection lens 36, and the measurement light source 37 formed from a near infrared LED with an invisible light wavelength, which also serves for measurement and alignment with respect to the eye E to be inspected, are arranged in the incident direction of the half mirror 32. A fixation target light source 38 formed from an LED for visual fixation of an object is arranged in the incident direction of the half mirror 35.

A pressure sensor 45 for monitoring the internal pressure of the air chamber, and a transfer tube 44 for transferring pressurized air from a cylinder 43 are connected to the inside of the air chamber 23. The transfer tube may have any form such as a bellows tube as shown in FIG. 2 or a metal tube. Alternatively, the cylinder 43 may be arranged so that it is directly connected to the air chamber 23 without using the transfer tube 44. A piston 40 is fitted in the cylinder 43, and driven by a solenoid 42. A drive lever 41 connected to the solenoid 42 and piston 40 converts a rotational motion of the solenoid 42 into a linear motion of the piston 40. In this system, as the piston 40 moves in the cylinder 43 at high speed, pressurized air in the cylinder 43 is sent to the air chamber 23 and puffed against the eye E to be inspected through the nozzle 22.

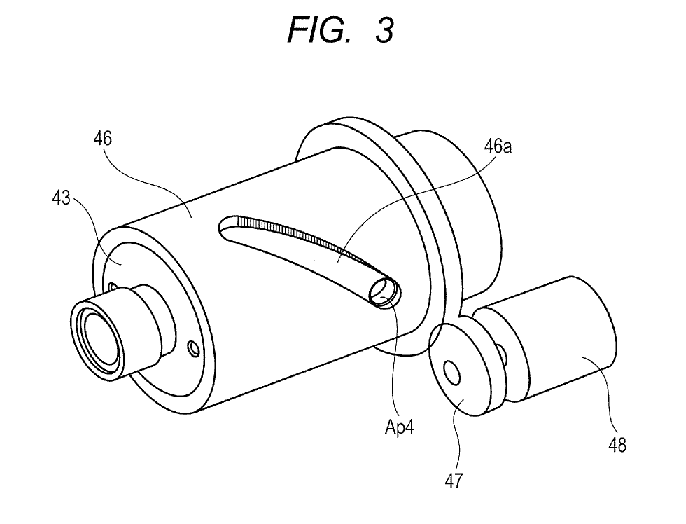

To practice the present invention, a plurality of air vent holes Ap1 to Ap4 for deciding the volume of pressurized air are formed in the outer wall of the cylinder 43. A rotation member 46 for selecting the air vent holes Ap1 to Ap4 is arranged outside the cylinder 43 so that the cylinder 43 is fitted in the rotation member 46. FIG. 3 is a view showing the outer appearance of the cylinder portion. The flange portion of the rotation member 46 has a gear shape and is arranged to engage with a drive gear 47 fitted on the output shaft of a drive motor, i.e., an air vent hole selection motor 48 in the embodiment. The rotation member 46 has a through hole portion 46a extending outside from the inside of a cylindrical shape in which the cylinder 43 is fitted. The rotation member 46 can be rotated by the drive gear 47 about the extension axis of the cylinder 43. In this example, the through hole portion 46a is an elongated hole which extends from the internal space of the rotation member 46 to the external space and crosses the extension axes of the cylinder 43 and rotation member 46.

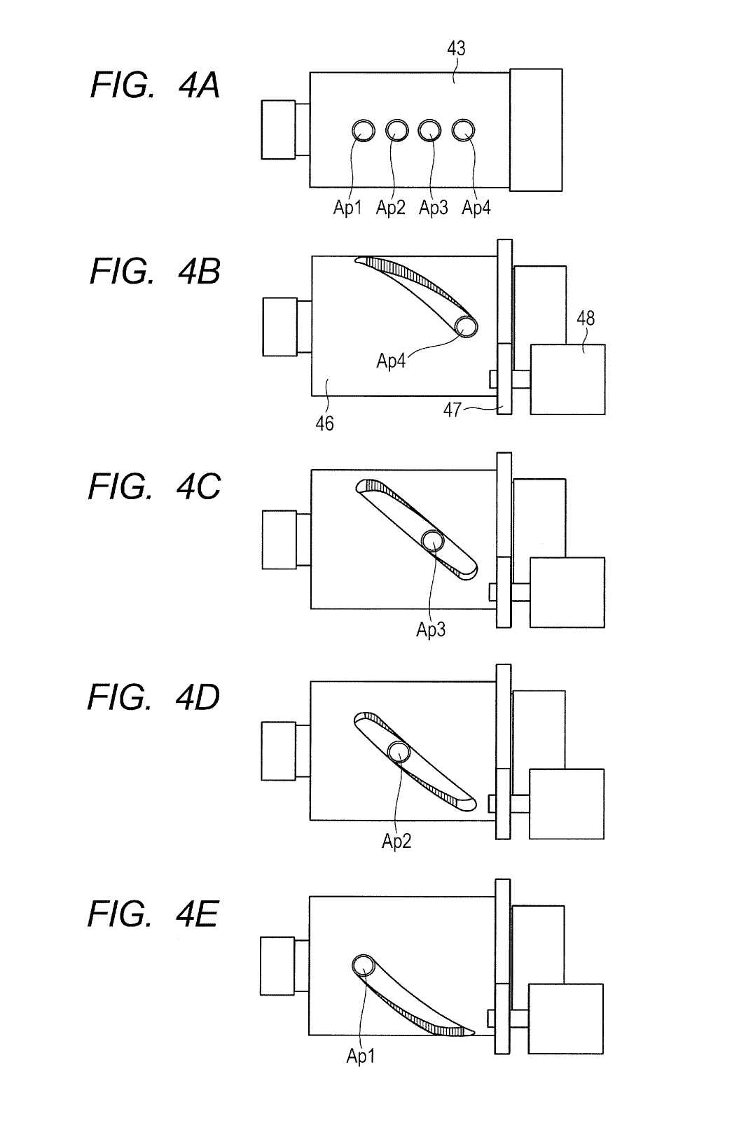

FIGS. 4A to 4E are views for explaining the operations of the cylinder 43 and rotation member 46. As shown in FIG. 4A, the air vent holes Ap1 to Ap4 are formed in the outer wall of the cylinder. The air vent hole selection motor 48 rotates to rotate the rotation member 46 via the drive gear 47. Along with this, the air vent hole connecting with the elongated hole 46a shifts sequentially, thereby selecting the air vent holes Ap1 to Ap4 capable of connecting the inside of the cylinder with the external space. The air vent hole Ap4 is selected in FIG. 4B, the air vent hole Ap3 is selected in FIG. 4C, the air vent hole Ap2 is selected in FIG. 4D, and the air vent hole Ap1 is selected in FIG. 4E. When each air vent hole is selected, the rotation member 46 closes an air vent hole arranged on the side of an eye to be inspected from the selected air vent hole, and an air vent hole arranged on the apparatus side. Note that the hole formed to extend from the internal space of the rotation member 46 to the external space is not limited to the above-mentioned elongated hole shape. The through hole portion 46a may be configured so that air vent holes connectable with the outside increase sequentially from, e.g., only the air vent hole Ap4 to the air vent holes Ap4 and Ap3, the air vent holes Ap4, Ap3, and Ap2, and the air vent holes Ap4, Ap3, Ap2, and Ap1 as the rotation member 46 rotates.

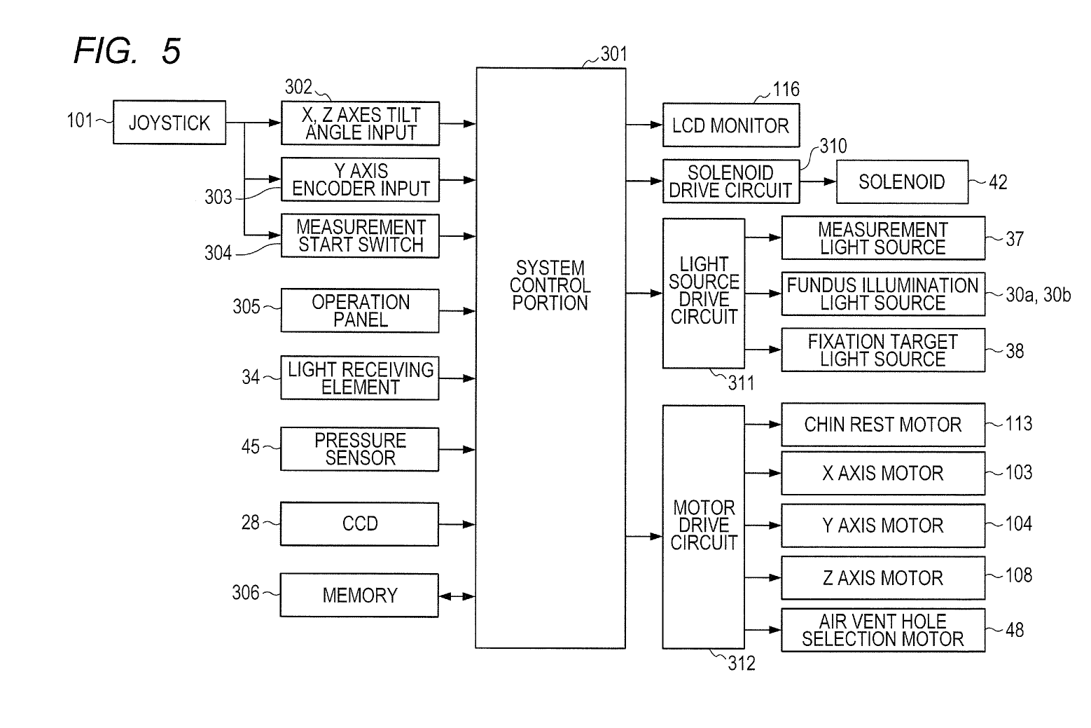

FIG. 5 is a system block diagram. A system control portion 301 which controls the overall system includes a program storage portion, a data storage portion which stores data for correcting an eye pressure value, an input/output control portion which controls input/output from/to various devices, and an arithmetic processing portion which calculates data obtained from various devices.

A tilt angle input 302 upon tilting the joy stick 101 back, forth, left, and right, an encoder input 303 upon rotating it, and a measurement start button input 304 upon pressing a measurement start button are connected to the system control portion from the joy stick 101 for positioning the measurement portion 110 with respect to the eye E to be inspected and starting measurement.

A print button, chin rest up and down buttons, and the like are arranged on an operation panel 305 on the base 100 (not shown). Upon receiving a button input, the system control portion is notified of the signal.

A memory 306 stores the anterior ocular segment image of the eye E to be inspected that is captured by the CCD 28. The reflection images of the pupil and cornea of the eye E to be inspected are extracted from the image stored in the memory 306 to perform alignment detection. The anterior ocular segment image of the eye E to be inspected that is captured by the CCD 28 is combined with text data and graphic data to display the anterior ocular segment image, measurement values, and the like on the LCD monitor 116.

The memory 306 stores a corneal shape change signal received by the light receiving element 34, and a signal from the pressure sensor 45 arranged in the air chamber 23.

The X axis motor 103, Y axis motor 104, Z axis motor 108, chin rest motor 113, and air vent hole selection motor 48 are driven in accordance with commands from the system control portion 301 via a motor drive circuit 312. The ON/OFF states of the measurement light source 37, fundus illumination light sources 30a and 30b, and fixation target light source 38, and change of the light amounts of them are controlled in accordance with commands from the system control portion 301 via a light source drive circuit 311.

The solenoid 42 is controlled by a signal from the system control portion 301. A change of the drive current and a voltage application ON/OFF operation are performed via a solenoid drive circuit 310.

In the embodiment, a rotary solenoid is used as the solenoid 42. The rotary solenoid is designed so that a movable pin moves in a copper wire-wound coil upon applying a voltage, and a mechanical component such as a bearing converts the linear motion into a rotational motion. Since the rotation torque is restricted in a unique direction, the solenoid returns to the initial position by a built-in coil spring.

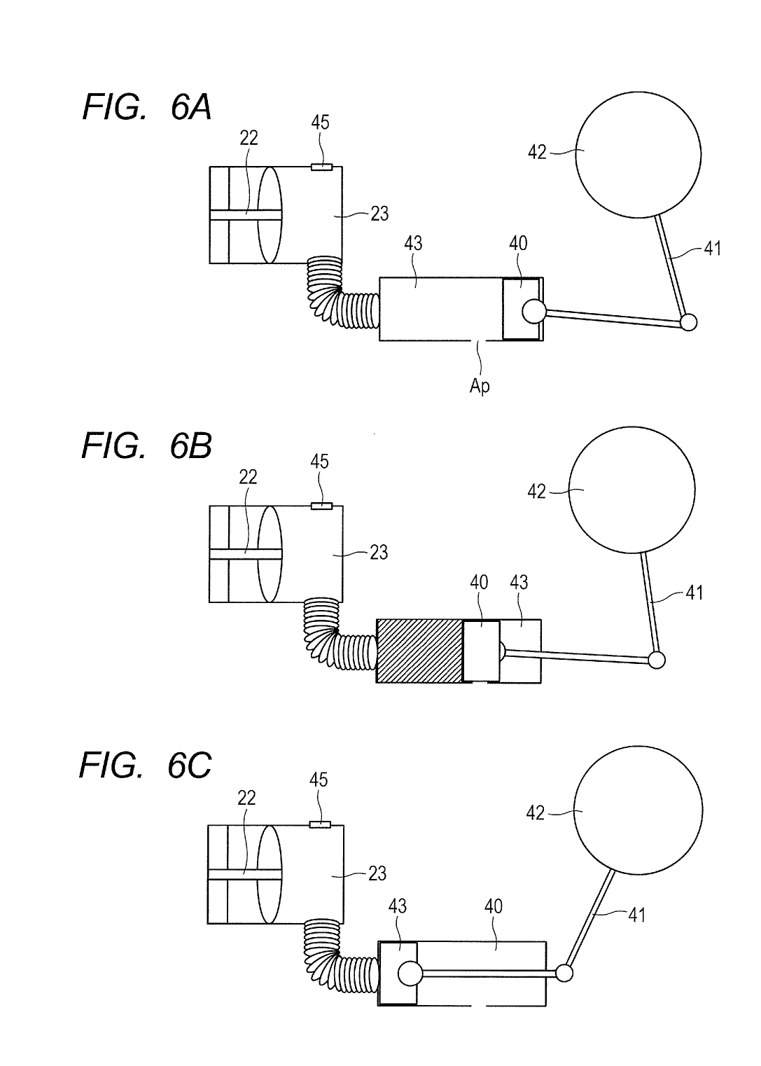

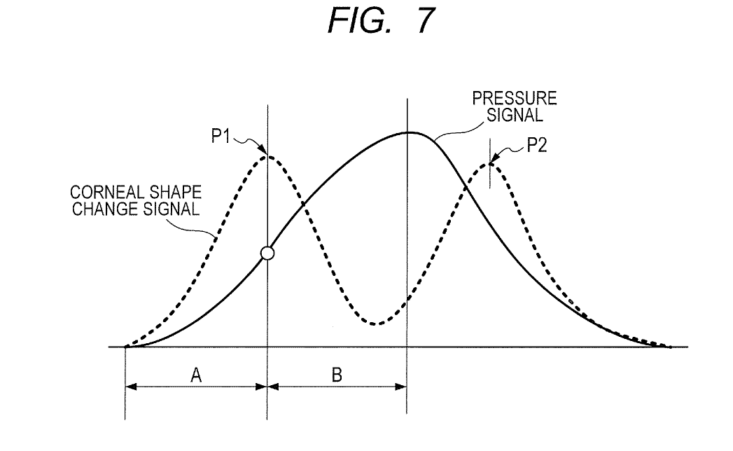

Prior to a detailed description of the present invention, solenoid control by the system control portion 301 in conventional eye pressure measurement will be described with reference to FIGS. 6A to 6C and 7. FIGS. 6A to 6C are views showing only an air puffing unit extracted from the optical arrangement view of FIG. 2. Each of FIGS. 6A to 6C shows the energization state of the solenoid 42 and the position of the piston 40 at that time. FIG. 7 shows the relationship between a pressure signal in the air chamber 23 that is obtained by the pressure sensor 45 upon eye pressure measurement, and the shape change state (to be referred to as a corneal shape change signal hereinafter) of the eye E to be inspected that is detected by the light receiving element 34. In FIG. 7, the abscissa represents the time after the start of measurement, and the ordinate represents the level of each signal.

A period A shown in FIG. 7 is a period during which the solenoid 42 is energized, and corresponds to a state change from FIG. 6A to FIG. 6B. Similarly, a period B shown in FIG. 7 is a state in which the drive current of the solenoid 42 is stopped, and corresponds to a state change from FIG. 6B to FIG. 6C.

FIG. 6A shows a piston position immediately before the start of energizing the solenoid 42. The piston 40 is fixed to a cylinder start end serving as an initial position by the torque of the coil spring incorporated in the solenoid 42. An air vent hole Ap is formed in the outer wall of the cylinder 43. When alignment between an eye to be inspected and the apparatus ends and eye pressure measurement starts, the system control portion 301 drives the solenoid 42 at high speed to move straight the piston 40 in the cylinder 43 at high speed. After the piston 40 passes the air vent hole Ap, it pressurizes air in the air chamber 23. As the internal pressure of the air chamber 23 rises, the air is puffed from the nozzle 22 against the cornea Ec of the eye E to be inspected, starting applanation of the cornea Ec.

As described above, the amount of light entering the light receiving element 34 is designed to be maximum at the instant of applanation of the cornea Ec by the puffed air. A point P1 at which the corneal shape change signal is maximized in FIG. 7 indicates the instant at which the cornea Ec changes from convexity to concavity. Upon detecting the maximum value of the corneal shape change signal, the system control portion 301 stops the drive current of the solenoid 42, and computes the eye pressure value of the eye E to be inspected from a simultaneously input pressure signal value indicated by a circle in FIG. 7.

The eye pressure values of healthy eyes generally range from 10 to 20 mmHg, and it is known that an eye suffering an eye disease such as glaucoma has a high eye pressure value of 20 mmHg or more. To cope with this, the apparatus needs to have a wide measurement range from about 0 to 60 mmHg. The position of the air vent hole Ap which is formed in the outer wall of the cylinder 43 to decide the volume of air to be pressurized, and the acceleration speed of the piston 40 are designed so that a maximum eye pressure value can be measured. In other words, the volume of pressurized air decided by the air vent hole Ap is too large for an eye to be inspected with a general eye pressure value equal to or smaller than the maximum eye pressure value.

In conventional measurement, therefore, it has been controlled to decrease the drive current of the solenoid 42 and advance the timing to stop the drive current, thereby decreasing unnecessary air puffed against an eye to be inspected.

However, it is known that the piston 40 has inertial force generated by its own weight and keeps moving even after the drive current of the solenoid 42 is stopped.

FIG. 6B shows a position of the piston 40 instantaneously when the point P1 in FIG. 7 is detected. FIG. 6C shows a position where the piston 40 finally stops. Even after the drive current is stopped, the piston 40 moves from the position in FIG. 68 to the position in FIG. 6C by the inertial force while keeping almost the same speed, and pressurizes the residual air in the cylinder 43 that is indicated by hatching in FIG. 6B. As a result, the pressurized air is puffed as air unnecessary for measurement against the eye to be inspected. The period B shown in FIG. 7 represents the relationship between the corneal shape change signal and the pressure signal when the piston 40 moves owing to the inertial force. It is known that even after the drive current of the solenoid 42 is stopped, the piston 40 keeps pressurizing air in the cylinder 43, and the pressure in the air chamber 23 keeps rising. The air puffed from the nozzle 22 changes the cornea Ec from the applanation state to the concave state, and the corneal shape change signal value decreases.

After the piston 40 stops in the state of FIG. 6C, it returns to the cylinder start end serving as the initial position shown in FIG. 6A by the torque of the coil spring incorporated in the solenoid 42.

Note that after puffing of air is stopped, the cornea Ec returns from the concave state to the normal convex state through the applanation state. At this time, the corneal shape change signal has a second peak point P2 as shown in FIG. 7.

The embodiment has described that the drive current of the solenoid 42 is stopped upon detecting the maximum value of the corneal shape change signal because the timing to stop the drive current of the solenoid 42 is not important. Although a detailed description will be omitted, if the peak value of the corneal shape change signal can be detected, the drive current of the solenoid 42 may be stopped instantaneously when, for example, the corneal shape change signal or pressure signal exceeds a predetermined threshold.

As already described above, the conventional non-contact tonometer has the serious problem that air unnecessary for measurement is puffed against an eye to be inspected owing to the inertial force of the piston 40 because the cylinder 43 is designed with reference to the maximum eye pressure. The present invention solves this problem by, if necessary, selecting a plurality of air vent holes formed in the outer wall of the cylinder 43 to change (decrease) the volume of air pressurized in the cylinder 43.

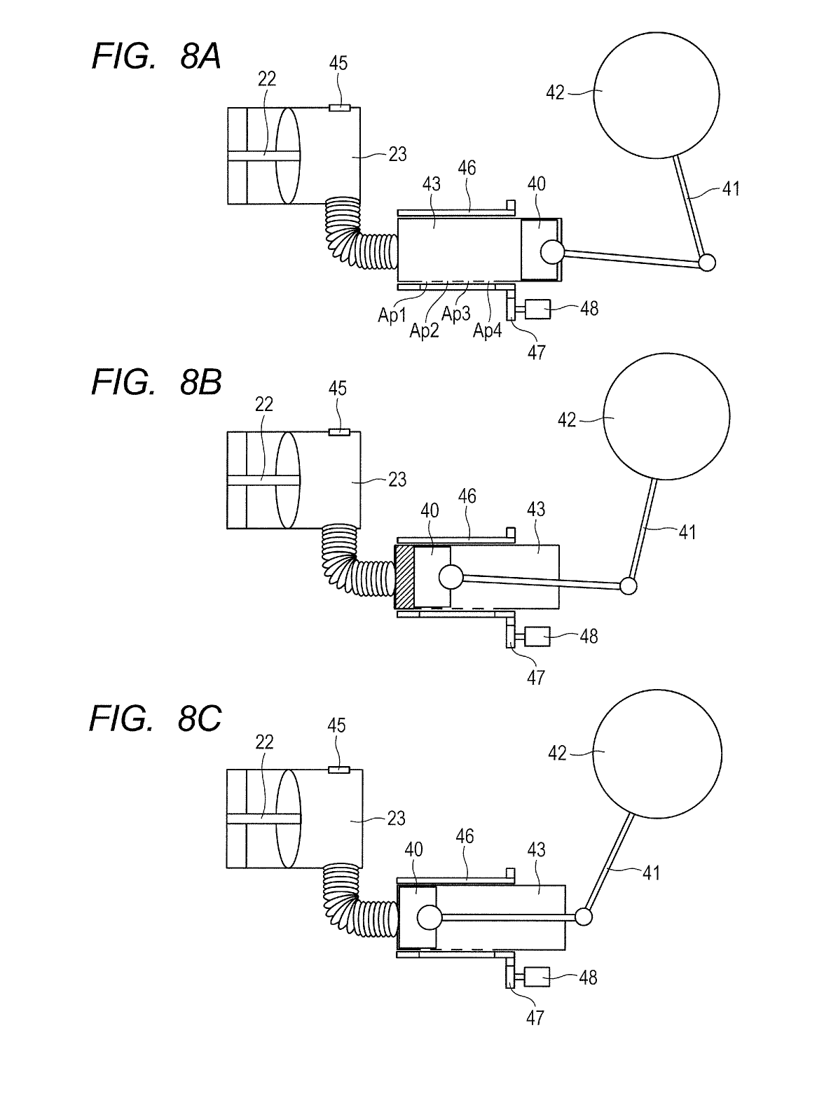

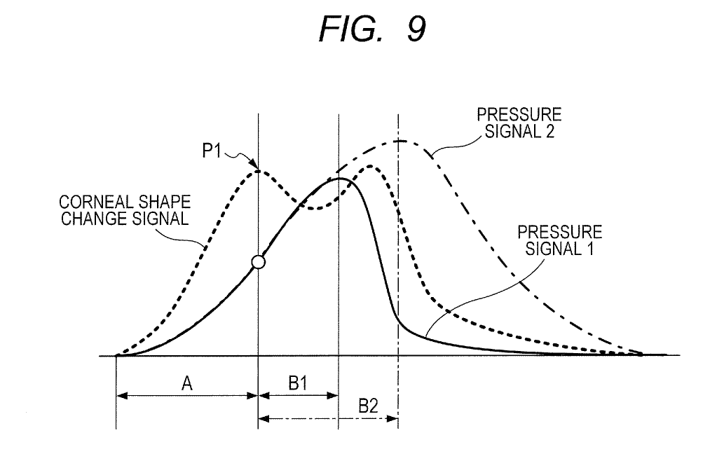

Next, the present invention will be described in detail with reference to FIGS. 8A to 8C and 9.

FIGS. 8A to 8C are views showing only an air puffing unit extracted from the optical arrangement view of FIG. 2. Each of FIGS. 8A to 8C shows the energization state of the solenoid 42 and the position of the piston 40 at that time. FIG. 9 shows the relationship between a pressure signal in the air chamber 23 that is obtained by the pressure sensor 45 upon eye pressure measurement, and the corneal shape change signal detected by the light receiving element 34. The abscissa represents the time elapsed after the start of measurement, and the ordinate represents the level of each signal. As in FIG. 7, a dotted line represents a corneal shape change signal, and a solid line represents a pressure signal (pressure signal 1) in the present invention. For comparison, a chain line represents a pressure signal (pressure signal 2) in the conventional control method.

FIG. 8A shows the operation start position of the piston 40 in the present invention. Unlike the arrangement in FIG. 6, a plurality of air vent holes (Ap1 to Ap4) described above are arranged, and can be selected by the air vent hole position selection rotation member 46 mentioned above. The air vent holes are set at optimal positions necessary to obtain a predetermined maximum eye pressure value. For example, the volume of the cylinder 43 necessary to measure an eye to be inspected with a maximum eye pressure of 20 mmHg can be easily computed by calculation. By arranging an air vent hole at a position where a cylinder volume computed by calculation is obtained, a measurement system in which the maximum eye pressure of 20 mmHg is set as an upper limit can be configured. In this configuration, for example, when four air vent holes are arranged, an air vent hole position can be selected in accordance with the eye pressure value of an eye to be inspected, such as a maximum eye pressure value of 20 mmHg, 30 mmHg, 40 mmHg, or 60 mmHg.

A case in which the air vent hole Ap1 is selected will be exemplified. After measurement starts, the solenoid 42 is driven at high speed in a period A shown in FIG. 9, similar to the conventional control. When the solenoid 42 is driven, the piston 40 moves in the cylinder 43 at high speed. After the piston 40 passes the air vent hole Ap1, air in the air chamber 23 is pressurized to raise the pressure signal. Applanation of the cornea Ec starts by puffing the air from the nozzle 22, and the corneal shape change signal also starts rising.

When the eye pressure value of the eye to be inspected is smaller than a maximum eye pressure value (e.g., 20 mmHg) set by the air vent hole Ap1, the system control portion 301 detects the corneal shape change signal peak value P1 before the piston 40 which has started from the position in FIG. 8A reaches the terminal end of the cylinder 43 shown in FIG. 8C (FIG. 9).

After obtaining the corneal shape change signal peak P1, the system control portion 301 stops the drive current of the solenoid 42, and computes the eye pressure value of the eye E to be inspected from a simultaneously input pressure signal value indicated by a circle in FIG. 9.

FIG. 8B shows a piston position instantaneously when the corneal shape change signal peak P1 is obtained. As described in the conventional control, the piston 40 keeps moving to the position in FIG. 8C, which is the terminal end of the cylinder 43, owing to the inertial force even after the drive current of the solenoid 42 is stopped.

However, the position where the piston 40 starts pressurizing air is changed because the air vent hole Ap1 is selected. Thus, the distance from the position in FIG. 8B to the position in FIG. 8C becomes shorter than that in the conventional control. As is obvious, the amount of residual air corresponding to a hatched portion shown in FIG. 8B is much smaller than that in the conventional measurement. In addition, a period B1 shown in FIG. 9, which corresponds to a period from the state in FIG. 83 to the state in FIG. 8C, that is, the time during which the piston 40 moves by the inertial force also becomes shorter than the moving time B2 in the conventional measurement.

As described above, the position where the piston 40 starts pressurizing air is changed by selecting an appropriate air vent hole in advance. By changing the volume of pressurized air in the cylinder 43, puffing of unnecessary air against an eye to be inspected can be suppressed, and an optimum amount of air can be puffed in accordance with the eye pressure value of the eye.

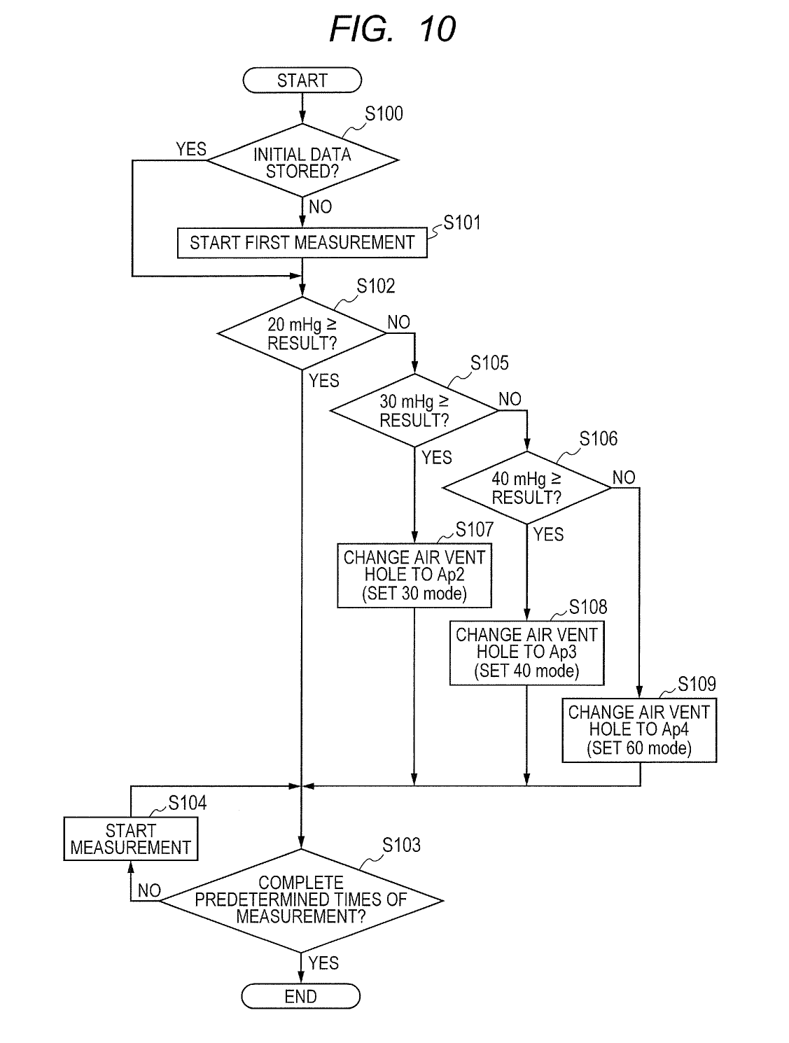

Finally, an example of the embodiment using the present invention will be described with reference to the flowchart of a measurement sequence in FIG. 10.

First, preparations before the start of measurement will be described briefly. The examiner instructs an object to rest his/her chin on the chin rest 112, and adjusts the eye to be inspected to a predetermined height in the Y axis direction by using the drive system 113. The examiner operates the joy stick 101 to a position where a cornea reflection image of the eye E to be inspected on the LCD monitor 116 is displayed.

If it is determined in step S100 that initial data of the object, i.e., existing eye pressure information measured in the past has been stored, an air vent hole position is selected based on this information (steps S102, S105, and S106). If the initial data of the object has not been stored, initial measurement starts (step S101).

As an initial setting, the above-mentioned Ap1 position is selected as the air vent hole position. The embodiment assumes that the position of the air vent hole Ap1 is set to be a position where the volume of pressurized air necessary to measure an eye to be inspected with a maximum eye pressure of 20 mmHg.

If the examiner presses the measurement start button in this state, automatic alignment starts first. In the alignment, a cornea bright spot formed by the cornea Ec is split by the prism stop 26, and is captured on the CCD 28 together with the eye F to be inspected, which is illuminated by the fundus illumination light sources 30a and 30b, and the bright spot images of the fundus illumination light sources 30a and 30b. The system control portion 301 stores the captured anterior ocular segment image of the eye E to be inspected in the memory 306, and performs alignment via the motor drive circuit 312 based on position information of respective bright spots extracted from the eye E to be inspected and the cornea reflection image. Upon completion of the alignment, the system control portion 301 drives the solenoid 42 and drives the piston 40 at high speed to measure the eye pressure.

In step S102, the system control portion 301 determines whether the measured eye pressure value is equal to or smaller than 20 mmHg. If the measured eye pressure value is equal to or smaller than 20 mmHg, the process shifts to step S103. In step S103, the system control portion 301 determines whether the measurement has been completed by predetermined times. If the measurement has not reached the predetermined times, the measurement is performed again in step S104. If the measurement has been completed by the predetermined times, the eye pressure measurement ends. If the number of predetermined times of measurement is one, the condition is satisfied by the measurement in step S101, so the eye pressure measurement ends without any more measurement.

If the measurement result is larger than 20 mmHg in step S102, the range where the eye pressure value of the eye to be inspected falls can be estimated from the peak and shape of a corneal shape change signal detected by the light receiving element 34 (steps S105 and S106). The system control portion 301 receives the corneal shape change signal from the light receiving element 34, compares it with eye pressure determination corneal shape change waveform data stored in advance in the memory 306, and estimates the range of the eye pressure value of the eye to be inspected by computation of the difference between them or the like. If the system control portion 301 estimates that the eye pressure value of the eye to be inspected falls within a range of 20 mmHg to 30 mmHg, it drives the air vent hole selection motor 48 via the motor drive circuit 312, and rotates the rotation member 46 via the drive gear 47 to change the air vent hole position to Ap2 (step S107) If the system control portion 301 estimates that the eye pressure value of the eye to be inspected falls within a range of 30 mmHg to 40 mm-Hg, it similarly changes the air vent hole position to Ap3 (step S108). If the system control portion 301 estimates that the eye pressure value of the eye to be inspected falls within a range of 40 mmHg or higher, it similarly changes the air vent hole position to Ap4 (step S109). After changing the air vent hole position, the process shifts to step S103 to execute eye pressure measurement till the completion of measurement by the predetermined times (step S104).

More specifically, in accordance with the measurement result of the eye pressure of an eye to be inspected by an eye pressure measuring unit, a pressurized gas volume change unit including the cylinder 43 and rotation member 46 changes the position where the inside of the cylinder 43 connects with the outside, thereby changing the substantial internal volume of the cylinder 43 used in gas pressurization.

After the end of the eye pressure measurement according to the above-described flowchart, control is performed according to a normal measurement routine including switching between the left and right eyes and printing of the measurement result. Then, all work ends.

As described above, the non-contact tonometer according to the present invention includes the corneal shape change unit which includes the piston 40 and cylinder 43, pressurizes a gas in the cylinder 43 by the piston 40, and puffs the pressurized gas against the cornea of an eye to be inspected from a nozzle accessory to the cylinder 43, thereby changing the corneal shape. The corneal shape change unit has opening portions each of which is formed in the outer wall of the cylinder 43, extends outside from the inside of the cylinder, and decides the internal volume of the cylinder when pressurizing a gas in the cylinder 43. Actual eye pressure measurement is performed by the eye pressure measuring unit which detects the state of a shape change of the cornea to measure the eye pressure of the eye to be inspected. Further, the non-contact tonometer includes the pressurized gas volume change unit which changes the position of the above-described opening portion at which the inside of the cylinder 43 can connect with the outside, thereby changing the internal volume of the cylinder when pressurizing the gas. A plurality of opening portions are preferably formed as in the embodiment. By selecting a predetermined one of the opening portions, the pressurized gas volume change unit changes the internal volume of the cylinder.

The embodiment has exemplified a case in which four air vent holes are used as a plurality of air vent holes, but the number of air vent holes is not limited to this. A plurality of air vent holes are arranged, and if necessary, the number of air vent holes can be increased/decreased.

### Second Embodiment

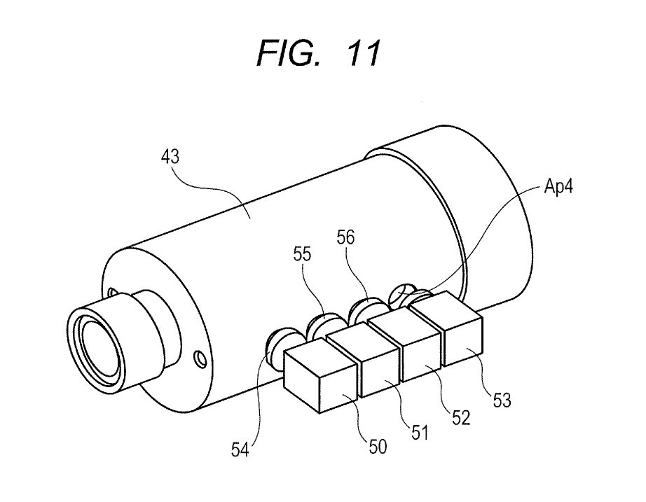

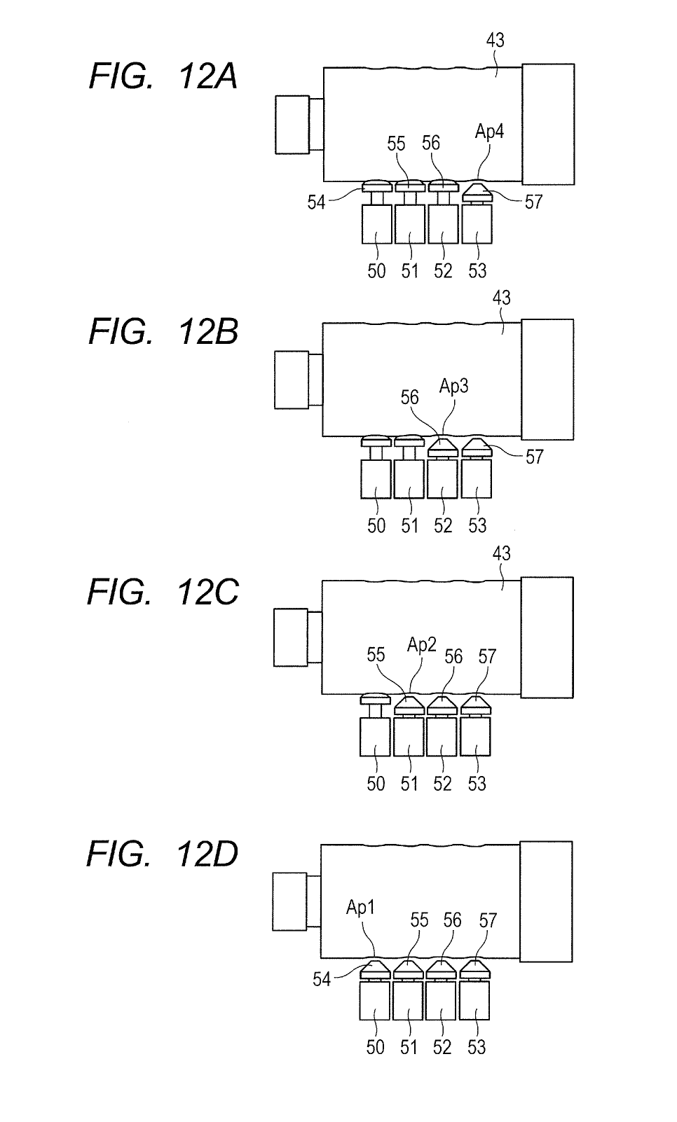

FIG. 11 is a view showing the outer appearance of a cylinder portion according to the second embodiment of the present invention. As in the first embodiment, a plurality of air vent holes Ap1 to Ap4 are formed in the outer wall of a cylinder 43. Electromagnetic valves 50 to 53, and stoppers 54 to 57 are arranged outside the cylinder 43 in correspondence with the respective air vent holes Ap1 to Ap4. The electromagnetic valves 50 to 53 can be independently driven. By driving the electromagnetic valves 50 to 53, the stoppers 54 to 57 can close the air vent holes Ap1 to Ap4. FIGS. 12A to 12D are views for explaining the operation of the cylinder portion in the embodiment. The air vent hole Ap4 is selected in FIG. 12A, the air vent hole Ap3 is selected in FIG. 12B, the air vent hole Ap2 is selected in FIG. 12C, and the air vent hole Ap1 is selected in FIG. 12D. When each air vent hole is selected, it is controlled to close an air vent hole arranged on the side of an eye to be inspected from the selected air vent hole, by an electromagnetic valve and stopper corresponding to the air vent hole.

By using the plurality of electromagnetic valves, an air vent hole can be selected in advance, as in the first embodiment. By changing the volume of pressurized air in the cylinder 43, puffing of unnecessary air against an eye to be inspected can be suppressed, and an optimum amount of air can be puffed in accordance with the eye pressure value of the eye to be inspected.

The embodiment has exemplified a case in which four air vent holes are used as a plurality of air vent holes. However, the number of air vent holes is not limited to this and can be increased/decreased, as needed.

### Third Embodiment

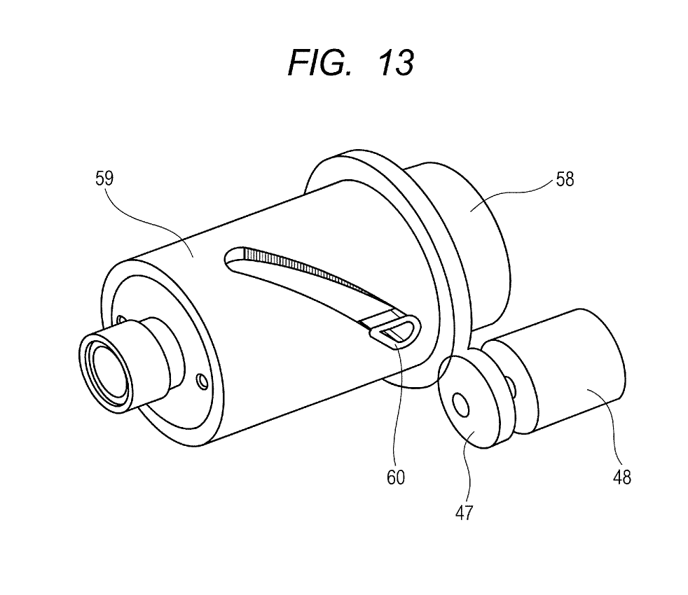

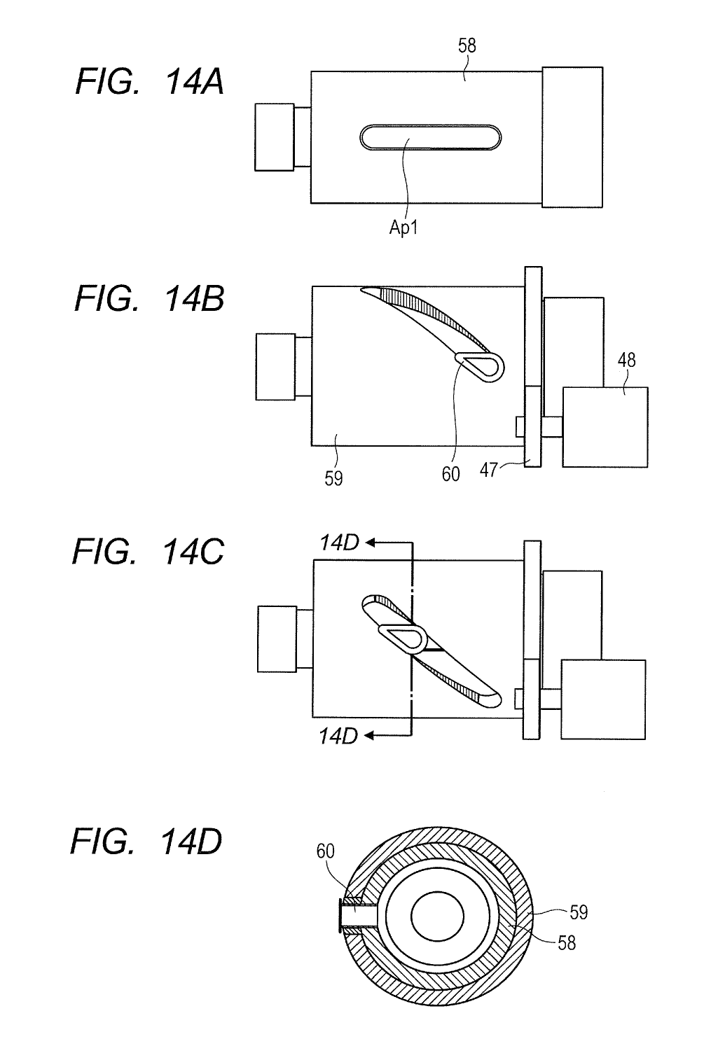

FIG. 13 is a view showing the outer appearance of a cylinder portion according to the third embodiment of the present invention. FIGS. 14A to 14D are views for explaining the operation of a cylinder portion in the third embodiment. As shown in FIG. 14A, an elongated air vent hole Ap1 is formed in the outer wall of a cylinder 58. A rotation member 59 for changing the air vent hole position is arranged outside the cylinder 58. An elongated hole is formed at an angle in the outer wall of the rotation member 59 so that it crosses the elongated hole of the cylinder 58. A leakage preventing member 60 is arranged at the intersection between the elongated air vent hole Ap1 of the cylinder 58 and the elongated hole of the rotation member 59 to prevent leakage of air from the groove of the elongated hole of the cylinder 58. Further, a drive gear 47 fitted on the output shaft of a drive motor 48 is arranged to mesh with the gear shape of the flange portion of the rotation member 59. The drive motor 48 rotates to rotate a rotation member 46 via the drive gear 47, thereby moving the leakage preventing member 60 along the shape of the air vent hole Ap1. FIG. 14B shows a case in which the air vent hole position is farthest from an eye to be inspected. FIG. 14C shows a case in which the air vent hole position comes close to the eye to be inspected. As shown in the sectional view of FIG. 14D, the central hole portion of the leakage preventing member 60 arranged at the intersection between the cylinder 58 and the rotation member 59 has the role of an air vent hole. Also, the outer wall of the leakage preventing member 60 has a role of preventing leakage of air from the elongated hole portion of the cylinder 58.

This structure can arbitrarily change the air vent hole position in advance. If necessary, the volume of pressurized air in a cylinder 43 can be changed without complicating the structure. Hence, puffing of unnecessary air against an eye to be inspected can be suppressed, and an optimum amount of air can be puffed in accordance with the eye pressure value of the eye.

### Another Embodiment 1

In eye pressure measurement by the related art shown in FIGS. 6A to 6C and 7, the position of the air vent hole Ap formed in the outer wall of the cylinder is fixed at a position where a maximum eye pressure of 60 mmHg can be measured, as described above. Even after the drive current of the solenoid 42 is stopped upon detecting the maximum value of a corneal shape change, the piston 40 keeps moving owing to the inertia and keeps pressurizing air in the cylinder 43 till the state of FIG. 6C. After the piston 40 stops in the state of FIG. 6C, it returns to the cylinder start end serving as the initial position shown in FIG. 6A by the torque of the coil spring incorporated in the solenoid 42. At this time, a problem occurs. For example, until the piston 40 passes the air vent hole Ap (close to the state of FIG. 6B), the pressure in the air chamber 23 becomes negative along with the piston movement to suck the tear of an eye to be inspected or the like via the nozzle 22.

By applying the structure described in each of the first to third embodiments according to the present invention to this phenomenon, the problem can be relieved. In the first to third embodiments, the air vent hole is arranged at a position closer to an eye to be inspected, compared to the conventional air vent hole position, and the drive system can change the air vent hole position. This will be explained by exemplifying the first embodiment. First, when performing initial eye pressure measurement, the air vent hole position Ap1 closest to the eye to be inspected is set, as described above. By driving the solenoid 42, the piston 40 moves in the cylinder 43 at high speed, and after passing the air vent hole Ap, pressurizes air in the air chamber 23, measuring an initial eye pressure. After the piston 40 stops in the state of FIG. 8C, the piston 40 starts moving toward the initial position of FIG. 8A by the torque of the coil spring incorporated in the solenoid 42 described above. At the same time, the pressure of air in the air chamber 23 starts changing to be negative. When the piston 40 passes the air vent hole Ap1, the negative pressure state in the air chamber 23 is canceled (close to the state of FIG. 8B). In the embodiment, compared to the conventional negative pressure cancellation position (close to the state of FIG. 6B), the negative pressure can be canceled at an early stage, and the problematic phenomenon such as suction of the tear of an eye to be inspected by the negative pressure can be suppressed.

As for the second and subsequent eye pressure measurements, the air vent hole position is changed in accordance with the eye pressure range of the eye to be inspected that has been turned out by the initial eye pressure measurement. Then, predetermined times of eye pressure measurement start. Similar to the initial eye pressure measurement, by driving the solenoid 42, the piston 40 moves in the cylinder 43 at high speed, and after passing the air vent hole, pressurizes air in the air chamber 23. As the internal pressure of the air chamber 23 rises, the air is puffed from the nozzle 22 against the cornea Ec of the eye E to be inspected, starting applanation of the cornea Ec. When the system control portion 301 detects the maximum value of the corneal shape change signal, it stops the drive current of the solenoid 42, and computes the eye pressure value of the eye E to be inspected from a simultaneously input pressure signal value indicated by the circle in FIG. 7. At the same time, the system control portion 301 rotates the air vent hole selection motor 48 via the motor drive circuit 312, and rotates the rotation member 46 via the drive gear 47, thereby moving the air vent hole to the position Ap1 closest to the eye to be inspected. By this operation, the negative pressure state of the air chamber 23 arising from the return operation of the piston 40 can be canceled quickly, and the problematic phenomenon such as suction of the tear of an eye to be inspected by the negative pressure can be suppressed. Even if the system according to the second or third embodiment is adopted, the problem can be relieved by a similar operation.

After the piston 40 stops in the state of FIG. 8C, the solenoid 42 can be driven again to hold the state of FIG. 8C. This can prevent even suction of the tear of an eye to be inspected by the negative pressure state of the air chamber 23.

In the first and second embodiments, a plurality of air vent holes are arranged in the axial direction of the cylinder 43, and one air vent hole exists in the circumferential direction. However, a plurality of air vent holes may exist in the circumferential direction. For the first embodiment, this can be implemented by forming, in the air vent hole selection rotation member 46, a hole shape configured to select an air vent hole for each hole formed in the circumferential direction in the cylinder 43. For the second embodiment, this can be implemented by arranging an electromagnetic valve and stopper for each hole in the circumferential direction. By arranging a plurality of holes in the circumferential direction, the above-mentioned negative pressure state can be canceled more quickly.

### Another Embodiment 2

The present invention is also implemented by executing the following processing. That is, this is the processing of supplying software (programs) for implementing the functions of the above embodiments to a system or apparatus via a network or various types of storage media and making the computer (or the CPU, MPU, or the like) of the system or apparatus read out and execute the software.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-086792, filed Apr. 17, 2013, which is hereby incorporated by reference herein in its entirety.

What is claimed is:

1. A non-contact tonometer including:
a corneal shape change unit configured to change a shape of a cornea of an eye to be inspected by pressurizing a gas in a cylinder by a piston and puffing the pressurized gas from a nozzle against the cornea, the corneal shape change unit including an opening portion configured to be formed in an outer wall of the cylinder and decide an internal volume of the cylinder when pressurizing the gas; and
an eye pressure measuring unit configured to detect a state of a shape change of the cornea and measure an eye pressure of the eye to be inspected, comprising:
a pressurized gas volume change unit configured to change a position where the opening portion can connect inside of the cylinder with outside, and change the internal volume of the cylinder when pressurizing the gas.

2. A tonometer according to claim 1, wherein the opening portion includes a plurality of opening portions, and the pressurized gas volume change unit selects a predetermined opening from the plurality of opening portions to change the connectable position.

3. A tonometer according to claim 1, wherein the opening portion includes a plurality of opening portions, and the pressurized gas volume change unit includes a plurality of electromagnetic valves configured to open/close each opening portion, and changes the connectable position by driving the plurality of electromagnetic valves.

4. A tonometer according to claim 2, wherein the plurality of opening portions are formed in an outer wall of the cylinder to make the internal volume of the cylinder when pressurizing the gas, which is decided by each opening portion, comply with a predetermined threshold when measuring the eye pressure of the eye to be inspected.

5. A tonometer according to claim 1, wherein
the pressurized gas volume change unit includes a rotation member configured to be fitted in the cylinder in a direction of an extension axis of the cylinder and be rotatable about the extension axis, and
the rotation member includes a through hole portion configured to extend from an internal space, in which the cylinder is fitted, to an external space, and connects the inside of the cylinder with the outside by aligning the through hole portion with the opening portion in a radial direction of the cylinder.

6. A tonometer according to claim 5, wherein the through hole portion includes an elongated hole configured to extend and cross the extension axis of the rotation member.

7. A tonometer according to claim 1, further comprising a control unit configured to control the pressurized gas volume change unit to change the connection position in accordance with a measurement result of the eye pressure of the eye to be inspected by the eye pressure measuring unit.

8. A method of measuring an eye pressure of an eye to be inspected, comprising the steps of:
pressurizing a gas in a cylinder by a piston;
puffing the pressurized gas against a cornea of the eye to be inspected from a nozzle to change the cornea; and
detecting a state of a shape change of the cornea to measure the eye pressure of the eye to be inspected,
wherein in the pressurizing step, a position where an opening portion configured to be formed in an outer wall of the cylinder and decide an internal volume of the cylinder when pressurizing the gas can connect inside of the cylinder with outside is changed to change the internal volume of the cylinder when pressurizing the gas.

9. A non-transitory tangible medium having recorded thereon a program for causing a computer to perform steps of the method according to claim 8.
